(19) United States
(12) Patent Application Publication
(10) Pub. No.: US 2026/0315498 A1
(43) Pub. Date: Oct. 15, 2026

(54) USE OF CANNABINOIDS IN THE TREATMENT OF EPILEPSY

(71) Applicant: JAZZ PHARMACEUTICALS RESEARCH UK LIMITED, SITTINGBOURNE (GB)

(72) Inventors: Geoffrey GUY, Cambridge (GB); Stephen WRIGHT, Altrincham (GB); Orrin DEVINSKY, New York, NY (US)

(21) Appl. No.: 19/304,483

(22) Filed: Aug. 19, 2025

Related U.S. Application Data
(63) Continuation of application No. 19/187,858, filed on Apr. 23, 2025, now Pat. No. 12,427,160.
Application No. 19/187,858 is a continuation of application No. 18/186,792, filed on Mar. 20, 2023, now Pat. No. 12,318,356.
Application No. 18/186,792 is a continuation of application No. 17/819,046, filed on Aug. 11, 2022, now Pat. No. 11,633,369.
Application No. 17/819,046 is a continuation of application No. 17/477,172, filed on Sep. 16, 2021, now Pat. No. 11,446,258.
Application No. 17/477,172 is a continuation of application No. 17/242,075, filed on Apr. 27, 2021, now Pat. No. 11,154,517.
Application No. 17/242,075 is a continuation of application No. 17/198,965, filed on Mar. 11, 2021, now Pat. No. 11,096,905.
Application No. 17/198,965 is a continuation of application No. 16/911,914, filed on Jun. 25, 2020, now Pat. No. 10,966,939.
Application No. 16/911,914 is a continuation of application No. 16/198,141, filed on Nov. 21, 2018, now Pat. No. 10,849,860.
Application No. 16/198,141 is a continuation of application No. 15/449,535, filed on Mar. 3, 2017, now Pat. No. 10,137,095.
Application No. 15/449,535 is a continuation of application No. 14/881,969, filed on Oct. 13, 2015, now Pat. No. 10,111,840.

(30) Foreign Application Priority Data
Oct. 14, 2014 (GB) 1418171.3

(51) Int. Cl. A61K 31/00; A61K 9/08; A61K 31/05; A61K 45/06; A61K 47/10

(52) U.S. Cl. CPC A61K 31/658; A61K 9/08; A61K 45/06; A61K 47/10

(57) ABSTRACT

The present disclosure relates to the use of cannabidiol (CBD) for the treatment of atonic seizures. In particular the CBD appears particularly effective in reducing atonic seizures in patients suffering with etiologies that include: Lennox-Gastaut Syndrome; Tuberous Sclerosis Complex; Dravet Syndrome; Doose Syndrome; Aicardi syndrome; CDKL5 and Dup15q in comparison to other seizure types. The disclosure further relates to the use of CBD in combination with one or more anti-epileptic drugs (AEDs).

### FIELD OF THE INVENTION

The present invention relates to the use of cannabidiol (CBD) in the treatment of atonic seizures. In one embodiment the patients suffering from atonic seizures are children and young adults. CBD appears particularly effective in reducing atonic seizures in patients suffering with etiologies that include: Lennox-Gastaut Syndrome; Tuberous Sclerosis Complex; Dravet Syndrome; Doose Syndrome; Aicardi syndrome; CDKL5 and Dup15q in comparison to other seizure types.

In these patients treatment with CBD reduced the occurrence of atonic seizures by greater than 50% in a large proportion, namely 63%, of patients. This was surprising given that the proportion of patients benefitting from a greater than 50% reduction in total seizures was significantly less, (48%), in all subjects treated.

Preferably the CBD used is in the form of a highly purified extract of Cannabis such that the CBD is present at greater than 98% of the total extract (w/w) and the other components of the extract are characterised. In particular the cannabinoid tetrahydrocannabinol (THC) has been substantially removed, to a level of not more than 0.15% (w/w) and the propyl analogue of CBD, cannabidivarin, (CBDV) is present in amounts of up to 1%. Alternatively, the CBD may be a synthetically produced CBD.

In use the CBD may be given concomitantly with one or more other anti-epileptic drugs (AED). Alternatively the CBD may be formulated for administration separately, sequentially or simultaneously with one or more AED or the combination may be provided in a single dosage form. Where the CBD is formulated for administration separately sequentially or simultaneously it may be provided as a kit or together with instructions to administer the one or more components in the manner indicated. It may also be used as the sole medication, i.e. as a monotherapy.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom from the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment-resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from an investigation into the type of seizures that the patient is experiencing is undertaken. Clinical observations and electroencephalography (EEG) tests are conducted and the type(s) of seizures are classified according to the ILEA classification described below and in FIG. 1.

The International classification of seizure types proposed by the ILAE was adopted in 1981 and a revised proposal was published by the ILAE in 2010 and has not yet superseded the 1981 classification. FIG. 1 is adapted from the 2010 proposal for revised terminology and includes the proposed changes to replace the terminology of partial with focal. In addition the term “simple partial seizure” has been replaced by the term “focal seizure where awareness/responsiveness is not impaired” and the term “complex partial seizure” has been replaced by the term “focal seizure where awareness/consciousness is impaired”.

From FIG. 1 it can be seen that Generalised seizures, where the seizure arises within and rapidly engages bilaterally distributed networks, can be split into six subtypes: Tonic-Clonic (grand mal) seizures; Absence (petit mal) Seizures; Clonic Seizures; Tonic Seizures; Atonic Seizures and Myoclonic Seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a bilateral convulsive seizure, which is the proposed terminology to replace Secondary Generalized Seizures (generalized seizures that have evolved from focal seizures and are no longer remain localized).

Focal seizures where the subject's awareness/responsiveness is altered are referred to as focal seizures with impairment and focal seizures where the awareness or responsiveness of the subject is not impaired are referred to as focal seizures without impairment.

Atonic seizures involve the loss of muscle tone, causing the person to fall to the ground. These are sometimes called ‘drop attacks’ and are usually brief (less than 15 seconds). Atonic seizures can occur without warning while standing, sitting and walking and the patient often suffers from trauma due to falling.

Atonic seizures are often associated with Lennox-Gastaut Syndrome but also occur, and may be symptomatic of other types of epileptic syndromes including: Tuberous Sclerosis Complex; Dravet Syndrome; Doose Syndrome; Aicardi syndrome; CDKL5 and Dup15q.

Epileptic syndromes often present with many different types of seizure and identifying the types of seizure that a patient is suffering from is important as many of the standard AED's are targeted to treat or are only effective against a given seizure type/sub-type.

One such childhood epilepsy syndrome is Lennox-Gastaut syndrome. Lennox-Gastaut syndrome is a severe form of epilepsy. Seizures usually begin before the age of 4. Seizure types, which vary among patients, include tonic (stiffening of the body, upward deviation of the eyes, dilation of the pupils, and altered respiratory patterns), atonic (brief loss of muscle tone and consciousness, causing abrupt falls), atypical absence (staring spells), and myoclonic (sudden muscle jerks). There may be periods of frequent seizures mixed with brief, relatively seizure-free periods.

Most children with Lennox-Gastaut syndrome experience some degree of impaired intellectual functioning or information processing, along with developmental delays, and behavioural disturbances.

Lennox-Gastaut syndrome can be caused by brain malformations, perinatal asphyxia, severe head injury, central nervous system infection and inherited degenerative or metabolic conditions. In 30-35 percent of cases, no cause can be found.

The first line treatment for atonic seizures, including the treatment of atonic seizures in patients with Lennox-Gastaut syndrome usually comprises a broad spectrum AED, such as sodium valproate often in combination with lamotrigine. Other AED that may be considered include rufinamide, felbamate, clobazam and topiramate.

AED such as carbamezapine, gabapentin, oxcarbazepine, pregabalin, tiagabineor and vigabatrin are contra-indicated in atonic seizures.

Common AED defined by their mechanisms of action are described in the following tables:

TABLE 1
Examples of narrow spectrum AED

| Narrow- | | |
|---|---|---|
| spectrum AED | Mechanism | Indication |
| Phenytoin | Sodium channel | Complex partial |
| | | Tonic-clonic |
| Phenobarbital | GABA/ | Partial seizures |
| | Calcium channel | Tonic-clonic |
| Carbamazepine | Sodium channel | Partial seizures |
| | | Tonic-clonic |
| | | Mixed seizures |
| Oxcarbazepine | Sodium channel | Partial seizures |
| | | Tonic-clonic |
| | | Mixed seizures |
| Gabapentin | Calcium channel | Partial seizures |
| | | Mixed seizures |
| Pregabalin | Calcium channel | Adjunct therapy for partial |
| | | seizures with or without |
| | | secondary generalisation |
| Lacosamide | Sodium channel | Adjunct therapy for partial |
| | | seizures |
| Vigabatrin | GABA | Secondarily generalized tonic- |
| | | clonic seizures |
| | | Partial seizures |
| | | Infantile spasms due to West |
| | | syndrome |

TABLE 2
Examples of broad spectrum AED

| Broad- | | |
|---|---|---|
| spectrum AED | Mechanism | Indication |
| Valproic acid | GABA/ | First-line treatment for tonic- |
| | Sodium | clonic seizures, absence |
| | channel | seizures and myoclonic |
| | | seizures |
| | | Second-line treatment for |
| | | partial seizures and infantile |
| | | spasms. |
| | | Intravenous use in status |
| | | epilepticus |
| Lamotrigine | Sodium | Partial seizures |
| | channel | Tonic-clonic |
| | | Seizures associated with |
| | | Lennox-Gastaut syndrome |
| Topiramate | GABA/ | Seizures associated with |
| | Sodium | Lennox-Gastaut syndrome |
| | channel | |
| Zonisamide | GABA/ | Adjunctive therapy in adults |
| | Calcium/ | with partial-onset seizures |
| | Sodium | Infantile spasm |
| | channel | Mixed seizure |
| | | Lennox-Gastaut syndrome |
| | | Myoclonic |
| | | Generalised tonic-clonic |
| | | seizure |
| Levetiracetam | Calcium | Partial seizures |
| | channel | Adjunctive therapy for partial, |
| | | myoclonic and tonic-clonic |
| | | seizures |
| Clonazepam | GABA | Typical and atypical absences |
| | | Infantile myoclonic |
| | | Myoclonic seizures |
| | | Akinetic seizures |
| | | Atonic seizures |
| Rufinamide | Sodium | Adjunctive treatment of partial |
| | channel | seizures associated with |
| | | Lennox-Gastaut syndrome |

TABLE 3
Examples of AED used specifically in childhood epilepsy

| AED | Mechanism | Indication |
|---|---|---|
| Clobazam | GABA | Adjunctive therapy in complex |
| | | partial seizures |
| | | Status epilepticus |
| | | Myoclonic |
| | | Myoclonic-absent |
| | | Simple partial |
| | | Complex partial |
| | | Absence seizures |
| | | Lennox-Gastaut syndrome |
| Stiripentol | GABA | Severe myoclonic epilepsy in |
| | | infancy (Dravet syndrome) |

From these tables it can be seen that there is only one drug currently approved for use in the treatment of atonic seizures, namely clonazepam. This medication works by the GABA mechanism.

Over the past forty years there have been a number of animal and human studies on the use of the non-psychoactive cannabinoid cannabidiol (CBD) to treat seizures.

A study in 1978 provided 200 mg/day of pure CBD to four adult patients, two of the four patients became seizure free, whereas in the remainder, seizure frequency was unchanged (Mechoulam and Carlini, 1978).

Cunha et al. reported that administration of CBD to eight adult patients with generalized epilepsy resulted in a marked reduction of seizures in 4 of the patients (Cunha et al., 1980) and Consroe et al., (1982) determined that CBD was able to prevent seizures in mice after administration of pro-convulsant drugs or an electric current.

In contrast to the studies described above, an open label study reported that 200 mg/day of pure CBD was ineffective in controlling seizures in twelve institutionalized adult patients (Ames and Cridland, 1986).

All of the studies described above focused on the treating subjects suffering from generalised epilepsy and did not look at the treatment of specific seizure sub-types.

More recently, WO 2011/001169 describes the use of CBD in the treatment of focal seizures, WO 2012/093255 describes the use of CBD in combination with standard anti-epileptic drugs in the treatment of epilepsy and WO 2013/045891 describes a composition comprising CBD and CBDV for use in the treatment of epilepsy.

In November 2013 the company GW Pharmaceuticals made a press release to state that they were intending to treat Dravet Syndrome with CBD as it had received orphan drug designation. The company made a further press release in February 2014 that that they were intending to treat Lennox-Gastaut Syndrome with CBD as it had also received orphan drug designation.

Again the rationale was to treat a disease as opposed to the type of seizure that the subject experienced.

It has additionally been suggested that Cannabis which is enriched in CBD may be efficacious in the treatment of epilepsy. A case study of a child with Lennox-Gastaut syndrome showed improvement in seizure frequency after treatment with CBD in an oily solution was reported in 2005 (Pelliccia et al. 2005).

Porter and Jacobson (2013) report on a parent survey conducted via a Facebook group which explored the use of Cannabis which was enriched with CBD in children with treatment-resistant epilepsy. It was found that sixteen of the 19 parents surveyed reported an improvement in their child's epilepsy. The children surveyed for this paper were all taking Cannabis that was purported to contain CBD in a high concentration although the amount of CBD present and the other constituents including THC were not known for many of the cases. Indeed, whilst CBD levels ranged from 0.5 to 28.6 mg/kg/day (in those extracts tested), THC levels as high as 0.8 mg/kg/day were reported. Providing children with TRE with a Cannabis extract that comprises THC, which has been described as a pro-convulsant (Consroe et al., 1977), at a potentially psychoactive dose of 0.8 mg/kg/day, is a concern.

In addition a paper published in June 2014 describes the use of a high-CBD strain to treat a patient with Dravet Syndrome; the patient's seizure frequency was stated to be reduced by the treatment (Maa et al. 2014).

A document published after the priority application was filed discloses the use of CBD in the treatment of refractory epilepsy in the treatment of Tuberous Sclerosis Complex in patients having focal onset seizures (Geffrey et al., 2014).

Whilst the potential of Cannabis and the cannabinoids, including CBD, to treat epilepsy has been rekindled, to date there has been little in the way of real data to support its efficacy in patients.

The applicant has found that CBD shows significant efficacy in reducing atonic seizures, by greater than 50% in a large proportion, namely 63%, of patients. By way of comparison the proportion of patients benefitting from a greater than 50% reduction in total seizures was significantly less, (46%), in all subjects treated.

It is additionally worth noting that the patients being treated were treatment resistant to existing AED and so consequently these figures are even the more remarkable.

### BRIEF SUMMARY OF THE DISCLOSURE

In accordance with a first aspect of the present invention there is provided cannabidiol (CBD) for use in the treatment of atonic seizures.

Preferably the atonic seizures are treatment-resistant.

Preferably the atonic seizures associated with Lennox-Gastaut Syndrome; Tuberous Sclerosis Complex; Dravet Syndrome; Doose Syndrome; Aicardi syndrome, CDKL5 or Dup15q.

In one embodiment the CBD is for use in combination with one or more concomitant anti-epileptic drugs (AED).

In a further embodiment the CBD is present as a highly purified extract of Cannabis which comprises at least 95% (w/w) CBD, more preferably 98% (w/w) CBD. Preferably the extract comprises less than 0.15% THC. More preferably the extract further comprises up to 1% CBDV.

In an alternative embodiment the wherein the CBD is present as a synthetic compound.

In a further embodiment of the invention the one or more AED is selected from the group consisting of: clobazam; clonazepam, levetiracetam; topiramate; stiripentol; phenobarbital; lacsamide; valproic acid; zonisamide; perampanel; and fosphenytoin.

Preferably the number of different anti-epileptic drugs that are used in combination with the CBD is reduced. Alternatively the dose of the one or more anti-epileptic drugs that are used in combination with the CBD is reduced.

Preferably the dose of CBD is greater than 5 mg/kg/day.

In accordance with a second aspect of the present invention there is provided a method of treating atonic seizures comprising administering cannabidiol (CBD) to a subject.

In accordance with a third aspect of the present invention there is provided a composition for use in the treatment of atonic seizures characterised by atonic seizures comprising cannabidiol (CBD), a solvent, a co-solvent, a sweetener, and a flavouring.

Preferably the solvent is sesame oil, the co-solvent is ethanol, the sweetener is sucralose, the flavouring is strawberry flavour and the CBD is present at a concentration of between 25/mg/ml and 100 mg/ml.

More preferably the composition comprises cannabidiol (CBD) at a concentration of between 25 to 100 mg/ml, ethanol at a concentration of 79 mg/mi, sucralose at a concentration of 0.5 mg/ml, strawberry flavouring at a concentration of 0.2 mg/ml and sesame q.s. to 1.0 ml.

### BRIEF DESCRIPTION OF THE DRAWINGS

FIG. 1 shows the ILEA 2010 proposal for revised terminology for organization of seizures and epilepsies.

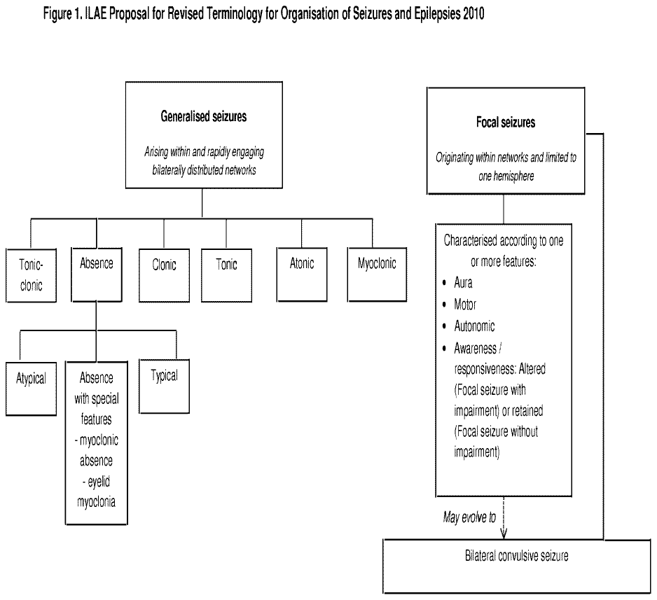

### DEFINITIONS

Definitions of some of the terms used to describe the invention are detailed below:

The cannabinoids described in the present application are listed below along with their standard abbreviations.

TABLE 4
Cannabinoids and their abbreviations

| | | |
|---|---|---|
| CBD | Cannabidiol | 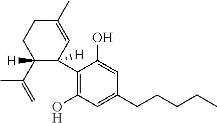 [SMILES: C=C(C)[C@@H]1CCC(C)=C[C@H]1c1c(O)cc(CCCCC)cc1O] |
| CBDA | Cannabidiolic acid | 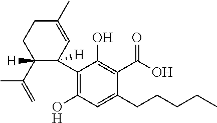 [SMILES: C=C(C)[C@@H]1CCC(C)=C[C@H]1c1c(O)cc(CCCCC)c(C(=O)O)c1O] |
| CBDV | Cannabidivarin | 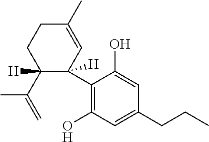 [SMILES: C=C(C)[C@@H]1CCC(C)=C[C@H]1c1c(O)cc(CCC)cc1O] |
| CBDVA | Cannabidivarinic acid | 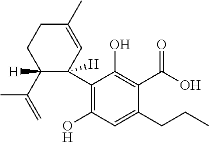 [SMILES: C=C(C)[C@@H]1CCC(C)=C[C@H]1c1c(O)cc(CCC)c(C(=O)O)c1O] |
| THC | Tetrahydro- cannabinol | 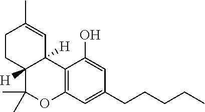 [SMILES: CCCCCc1cc(O)c2c(c1)OC(C)(C)[C@@H]1CCC(C)=C[C@@H]21] |

The table above is not exhaustive and merely details the cannabinoids which are identified in the present application for reference. So far over 60 different cannabinoids have been identified and these cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the Cannabis plant. The phytocannabinoids can be isolated from plants to produce a highly purified extract or can be reproduced synthetically.

“Highly purified cannabinoids” are defined as cannabinoids that have been extracted from the Cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example it is known that heating the carboxylic acid form will cause most of the carboxylic acid form to decarboxylate into the neutral form.

“Treatment-resistant epilepsy” (TRE) or “intractable epilepsy” is defined as per the ILAE guidance of 2009 as epilepsy that is not adequately controlled by trials of one or more AED.

“Childhood epilepsy” refers to the many different syndromes and genetic mutations that can occur to cause epilepsy in childhood. Examples of some of these are as follows: Dravet Syndrome: Myocionic-Absence Epilepsy; Lennox-Gastaut syndrome; Generalized Epilepsy of unknown origin; CDKL5 mutation. Aicardi syndrome; bilateral polymicrogyria; Dup15q; SNAP25; and febrile infection related epilepsy syndrome (FIRES); benign rolandic epilepsy; juvenile myoclonic epilepsy; infantile spasm (West syndrome); and Landau-Kleffner syndrome. The list above is non-exhaustive as many different childhood epilepsies exist.

“Atonic Seizures” are defined as a convulsive type of epileptic seizure which causes the muscles to relax and the patient to flop or fall.

“Mixed seizures” are defined as the existence of both generalised and focal seizures in the same patient.

The terms “50% responder” and “50% reduction in seizure” are both terms used in clinical studies. In the present application the terms define the percentage of subjects that experienced a greater than or equal to 50% reduction in the number of seizures during treatment with CBD in comparison to the number experienced during the baseline period before the CBD was administered.

### DETAILED DESCRIPTION

### Preparation of Highly Purified CBD Extract

The following describes the production of the highly-purified (>98% w/w) cannabidiol extract which has a known and constant composition which was used for the expanded access trials described in Examples below.

In summary the drug substance used in the trials is a liquid carbon dioxide extract of high-CBD containing chemotypes of Cannabis saliva L. which had been further purified by a solvent crystallization method to yield CBD. The crystallisation process specifically removes other cannabinoids and plant components to yield greater than 95% CBD w/w, typically greater than 98% w/w.

The Cannabis saliva L. plants are grown, harvested, and processed to produce a botanical extract (intermediate) and then purified by crystallization to yield the CBD (drug substance).

The plant starting material is referred to as Botanical Raw Material (BRM); the botanical extract is the intermediate; and the active pharmaceutical ingredient (API) is CBD, the drug substance.

Both the botanical starting material and the botanical extract are controlled by specifications. The drug substance specification is described in Table 5 below.

TABLE 5
CBD Specification

| Test | Test Method | Limits |
|---|---|---|
| Appearance | Visual | Off-white/pale yellow crystals |
| Identification A | HPLC-UV | Retention time of major peak |
| | | corresponds to certified CBD |
| | | Reference Standard |
| Identification B | GC-FID/MS | Retention time and mass |
| | | spectrum of major peak |
| | | corresponds to certified CBD |
| | | Reference Standard |
| Identification C | FT-IR | Conforms to reference |
| | | spectrum for certified CBD |
| | | Reference Standard |
| Identification D | Melting Point | 65-67 °C |
| Identification E | Specific Optical | Conforms with certified CBD |
| | Rotation | Reference Standard; −110° to |
| | | −140° (in 95% ethanol) |
| Total Purity | Calculation | ≥98.0% |
| Chromatographic Purity | HPLC-UV | ≥98.0% |
| 1 | | |
| Chromatographic Purity | GC-FID/MS | ≥98.0 % |
| 2 | | |
| Other Cannabinoids: | HPLC-UV | |
| CBDA | | NMT 0.15% w/w |
| CBDV | | NMT 1.0% w/w |
| Δ⁹ THC | | NMT 0.15% w/w |
| CBD-C4 | | NMT 0.5% w/w |
| Residual Solvents: | GC | |
| Alkane | | NMT 0.5% w/w |
| Ethanol | | NMT 0.5% w/w |
| Residual Water | Karl Fischer | NMT 1.0% w/w |

NMT-Not more than

The purity of the CBD drug substance achieved is greater than 98%. The other cannabinoids which may occur in the extract are: CBDA, CBDV, CBD-C4 and THC.

Distinct chemotypes of Cannabis sativa L. plant have been produced to maximize the output of the specific chemical constituents, the cannabinoids. One type of plant produces predominantly CBD. Only the (−)-trans isomer occurs naturally, furthermore during purification the stereochemistry of CBD is not affected.

### Production of the Intermediate

An overview of the steps to produce a botanical extract, the intermediate, are as follows:
1. Growing
2. Decarboxylation
3. Extraction No. 1—using liquid CO₂
4. Extraction No. 2—‘winterization’ using ethanol
5. Filtration
6. Evaporation

High CBD chemovars were grown, harvested and dried and stored in a dry room until required. The botanical raw material (BRM) was finely chopped using an Apex mill fitted with a 1 mm screen. The milled BRM was stored in a freezer for up to 3 months prior to extraction.

Decarboxylation of CBDA to CBD was carried out using a large Heraeus tray oven. The decarboxylation batch size in the Heraeus is approximately 15 Kg. Trays were placed in the oven and heated to 105° C.; the BRM took 96.25 minutes to reach 105° C. Held at 105° C. for 15 Minutes. Oven then set to 150° C.; the BRM took 75.7 minutes to reach 150° C.; BRM held at 150° C. for 130 Minutes. Total time in the oven was 380 Minutes, including 45 minutes cooling and 15 Minutes venting.

Extraction No 1 was performed using liquid CO₂ at 60 bar/10° C. to produce botanical drug substance (BDS) which was used for crystallisation to produce the test material.

The crude CBD BDS was winterised in Extraction No 2 under standard conditions (2 volumes of ethanol at minus 20° C. for around 50 hours). The precipitated waxes were removed by filtration and the solvent evaporated using the rotary evaporator (water bath up to 60° C.) to yield the BDS.

### Production of the Drug Substance

The manufacturing steps to produce the drug substance from the intermediate botanical extract are as follows:
1. Crystallization using C5-C12 straight chain or branched alkane
2. Filtration
3. Optional recrystallization from C5-C12 straight chain or branched alkane
4. Vacuum drying

Intermediate botanical extract (12 kg) produced using the methodology above was dispersed in C5-C12 straight chain or branched alkane (9000 ml, 0.75 vols) in a 30 litre stainless steel vessel.

The mixture was manually agitated to break up any lumps and the sealed container then placed in a freezer for approximately 48 hours.

The crystals were isolated by vacuum filtration, washed with aliquots of cold C5-C12 straight chain or branched alkane (total 12000 ml), and dried under a vacuum of <10 mb at a temperature of 60° C. until dry before submitting the drug substance for analysis. The dried product was stored in a freezer at minus 20° C. in a pharmaceutical grade stainless steel container, with FDA food grade approved silicone seal and clamps.

### Production of the Drug Product

The drug product is presented as an oral solution. The oral solution presentation contains 25 mg/ml or 100 mg/ml CBD, with the excipients sesame oil, ethanol, sucralose and flavouring. Two product strengths are available to allow dose titration across a wide dose range.

The 25 mg/ml solution is appropriate at lower doses and the 100 mg/ml solution at higher doses.

The drug product formulation is as described in Table 6 below:

TABLE 6
Drug Product specification

| | Qualitative | | Reference to |
|---|---|---|---|
| Component | Composition | Function | Quality Standard |
| Cannabidiol (CBD) | 25 mg/ml or | Active | In-house |
| | 100 mg/ml | | |
| Anhydrous ethanol | 79.0 mg/ml* | Excipient | Ph.Eur. |
| Sucralose | 0.5 mg/ml | Sweetener | In-house |
| Strawberry | 0.2 mg/ml | Flavouring | In-house |
| flavouring | | | |
| Sesame oil | q.s to 1.0 ml | Excipient | Ph.Eur. |

The drug substance, CBD is insoluble in water. Sesame oil was selected as an excipient to solubilize the drug substance.

A sweetener and fruit flavouring are required to improve palatability of the sesame oil solution.

Ethanol was required to solubilize the sweetener and the flavouring.

The composition can be substantially equivalent, by which is meant the functional ingredients can vary from the qualitative composition specified in Table 6 by an amount of up to 10%.

Example 1 below describes the use of a highly purified Cannabis extract comprising cannabidiol (CBD) in an expanded access treatment program in children with TRE.

### Example 1: Efficacy of Cannabidiol Reducing Atonic Seizures in Children and Young Adults with Intractable Epilepsy

### Materials and Methods

Of 137 children and young adults with severe, childhood onset treatment-resistant epilepsy (TRE), twenty-seven suffered from epilepsy that was characterised by atonic seizures. These subjects were tested with a highly purified extract of cannabidiol (CBD) obtained from a Cannabis plant. All subjects presented with atonic type seizures, often in addition to other seizures. The participants in the study were part of an expanded access compassionate use program for CBD.

The epileptic syndromes that these patients suffered from were as follows: Lennox-Gastaut Syndrome; Tuberous Sclerosis Complex; Dravet Syndrome: Doose Syndrome; Aicardi syndrome, CDKL5 and Dup15q.

All patients entered a baseline period of 4 weeks when parents/caregivers kept prospective seizure diaries, noting all countable seizure types.

The patients then received a highly purified CBD extract (greater than 98% CBD w/w) in sesame oil, of known and constant composition, at a dose of 5 mg/kg/day in addition to their baseline anti-epileptic drug (AED) regimen.

The daily dose was gradually increased by 2 to 5 mg/kg increments until intolerance occurred or a maximum dose of 25 mg/kg/day was achieved.

Patients were seen at regular intervals of 2-4 weeks. Laboratory testing for hematologic, liver, kidney function, and concomitant AED levels was performed at baseline, and after 4 weeks of CBD therapy.

All patients were taking at least two concomitant anti-epileptic drugs. These included clobazam; levetiracetam; topiramate; stiripentol; phenobarbital; lacsamide; valproic acid; zonisamide. The average number of concomitant antiepileptic drugs being taken was 2.7. The majority took either clobazam and/or valproic acid.

### Results

There were 27 children and young adult patients all of whom suffered from atonic seizures received treatment with CBD for at least 12 weeks.

A summary of the 50% responders, based on 12 weeks of treatment are summarized in Table 7 below.

TABLE 7
Summary of 50% responders after 12 weeks of treatment for
atonic seizures

| | Atonic seizures | Total seizures |
|---|---|---|
| | (n = 27) | (n = 137) |
| >50% reduction in | 63% (n = 17) | 46% (n = 63) |
| seizures | | |
| <50% reduction in | 37% (n = 10) | 54% (n = 74) |
| seizures | | |

Table 7 shows that after 3 months of therapy, a remarkable 63% of patients had an equal to or greater than >50% reduction in atonic seizures, these data infer that the CBD is very effective at reducing this type of seizure.

### CONCLUSIONS

These data indicate that CBD significantly reduces the number of atonic seizures in a high proportion of patients that do not respond well to existing AED.

It was surprising that in this group of patients which are treatment-resistant such a high number were able to gain an effect. The fact that nearly two thirds of the patients (63%) benefitted from at least a fifty percent reduction in the number of atonic seizures that they suffered from was remarkable.

Furthermore when these data are compared to the other sub-types of generalised seizure, it can clearly be seen that CBD was able to selectively reduce the occurrence of atonic seizures. Table 8 below details these findings.

TABLE 8
Summary of 50% responders after 12 weeks of treatment
for all seizure sub-types

| | | | Tonic- | | | |
|---|---|---|---|---|---|---|
| | Atonic | Tonic | clonic | Clonic | Myoclonic | Absence |
| | seizures | seizures | seizures | seizures | seizures | seizures |
| | (n = 27) | (n = 45) | (n = 65) | (n = 8) | (n = 30) | (n = 28) |
| >50% | 63% | 49% | 43% | 50% | 43% | 64% |
| reduction | (n = 17) | (n = 22) | (n = 28) | (n = 4) | (n = 13) | (n = 18) |
| in seizures | | | | | | |
| <50% | 37% | 51% | 37% | 50% | 57% | 36% |
| reduction | (n = 10) | (n = 23) | (n = 37) | (n = 4) | (n = 17) | (n = 10) |
| in seizures | | | | | | |

From Table 8 it can be seen that when the number of atonic seizures recorded is compared with other generalised seizure types such as tonic seizures (49% of patients experienced a greater than 50% reduction in seizure), tonic-clonic seizures (43% of patients experienced a greater than 50% reduction in seizure), and myoclonic seizures (43% of patients experienced a greater than 50% reduction in seizure) the fact that nearly two thirds (63%) of patients experiencing atonic seizures had a greater than 50% reduction in the number of seizures that occurred is very surprising.

### REFERENCES

Ames F R and Cridland S (1986). “Anticonvulsant effects of cannabidiol.” S Afr Med J 69:14.
Consroe P, Martin P, Eisenstein D. (1977). “Anticonvulsant drug antagonism of delta-9-tetrahydrocannabinol induced seizures in rabbits.” Res Commun Chem Pathol Pharmacol. 16:1-13
Consroe P, Benedicto M A, Leite J R. Carlini E A, Mechoulam R. (1982). “Effects of cannabidiol on behavioural seizures caused by convulsant drugs or current in mice.” Eur J Pharmaco. 83: 293-8
Cunha J M, Carlini E A, Pereira A E, Ramos O L, Pimental C, Gagliardi R et al. (1980). “Chronic administration of cannabidiol to healthy volunteers and epileptic patient.” Pharmacology. 21:175-85
Dravet C. The core Dravet syndrome phenotype. Epilepsia. 2011 April; 52 Suppl 2:3-9.
Eadie, MJ (December 2012). “Shortcomings in the current treatment of epilepsy.” Expert Review of Neurotherapeutics 12 (12): 1419-27.
Geffrey A, Pollack S, Paolini J, Bruno P, Thiele E (2014) “Cannabidiol (CBD) treatment for refractory epilepsy in Tuberous Sclerosis Complex (TSC).” American Epilepsy Society Annual Meeting. 5-9 Dec. 2014.
Kwan P, Arzimanoglou A, Berg A T, Brodie M J, Hauser W A, Mathem G, Moshé S L, Perucca E, Wiebe S, French J. (2009) “Definition of drug resistant epilepsy: Consensus proposal by the ad hoc Task Force of the ILAE Commission on Therapeutic Strategies.” Epilepsia.
Maa E and Figi P (2014). “The case for medical marijuana in epilepsy”, Epilepsia 55(6):783-786
Mechoulam R and Carlini E A (1978). “Toward drugs derived from cannabis.” Die naturwissenschaften 65:174-9.
Pelliccia A, Grassi G, Romano A, Crocchialo P (2005). “Treatment with CBD in oily solution of drug resistant paediatric epilepsies”. Congress of Cannabis and the Cannabinoids, Leiden, The Netherlands. International Association for Cannabis as a Medicine. p 14.
Porter B E, Jacobson C (December 2013). “Report of a parent survey of cannabidiol-enriched Cannabis use in paediatric treatment resistant epilepsy” Epilepsy Behaviour. 29(3) 574-7
Thurman, DJ; Beghi, E; Begley, CE; Berg, AT; Buchhalter, JR; Ding, D; Hesdorffer, DC; Hauser, WA; Kazis, L; Kobau, R: Kroner, B; Labiner, D; Liow, K; Logroscino, G; Medina, MT; Newton, CR; Parko, K; Paschal, A; Preux, P M; Sander, J W; Selassie, A; Theodore, W; Tomson, T; Wiebe, S; ILAE Commission on, Epidemiology (September 2011). “Standards for epidemiologic studies and surveillance of epilepsy.” Epilepsia. 52 Suppl 7: 2-26

1-30. (canceled)

31. A method of treating seizures in a patient with epilepsy and having a developmental delay, the method comprising orally administering to the patient a drug product comprising a CBD drug substance,
wherein the CBD drug substance has a purity of at least 98% (w/w) CBD, and
wherein the CBD is administered at a dose ranging from about 5 mg/kg/day to about 25 mg/kg/day.

32. The method of claim 31, wherein the epilepsy is a childhood epilepsy.

33. The method of claim 31, wherein the patient is a child.

34. The method of claim 31, wherein the epilepsy is associated with a genetic mutation.

35. The method of claim 31, wherein the developmental delay is associated with a genetic mutation.

36. The method of claim 31, wherein the epilepsy is treatment resistant.

37. The method of claim 31, wherein the developmental delay comprises a cognitive delay, a behavioral delay, a motor delay, or any combination thereof.

38. The method of claim 37, wherein the developmental delay comprises a cognitive delay.

39. The method of claim 37, wherein the developmental delay comprises a behavioral delay.

40. The method of claim 37, wherein the developmental delay comprises a motor delay.

41. The method of claim 31, wherein the epilepsy is associated with Dravet Syndrome, Lennox-Gastaut syndrome, CDKL5, Aicardi Syndrome, Dup15q, febrile infection related epilepsy syndrome, juvenile myoclonic epilepsy, West syndrome, Landau-Kleffner syndrome, or Doose Syndrome.

42. The method of claim 31, wherein the epilepsy is associated with Dravet Syndrome, Lennox-Gastaut syndrome, CDKL5 mutation, Aicardi Syndrome, or Dup15q.

43. The method of claim 31, wherein the CBD is present as a synthetic compound.

44. The method of claim 31, wherein the CBD is administered at a dose ranging from about 5 mg/kg/day to about 20 mg/kg/day.

45. The method of claim 31, wherein the CBD is administered at a dose ranging from about 10 mg/kg/day to about 20 mg/kg/day.

46. The method of claim 31, wherein the CBD is administered at a dose of about 10 mg/kg/day or about 20 mg/kg/day.

47. The method of claim 34, wherein the CBD is administered at a dose of about 10 mg/kg/day or about 20 mg/kg/day.

48. The method of claim 36, wherein the CBD is administered at a dose of about 10 mg/kg/day or about 20 mg/kg/day.

49. The method of claim 37, wherein the CBD is administered at a dose of about 10 mg/kg/day or about 20 mg/kg/day.

50. The method of claim 41, wherein the CBD is administered at a dose of about 10 mg/kg/day or about 20 mg/kg/day.
